(19) United States
(12) Patent Application Publication
(10) Pub. No.: US 2022/0007752 A1
(43) Pub. Date: Jan. 13, 2022

(54) EAR PROTECTOR HOOK

(71) Applicant: Robert Sason, Painesville, OH (US)

(72) Inventor: Robert Sason, Painesville, OH (US)

(21) Appl. No.: 16/925,731

(22) Filed: Jul. 10, 2020

(51) Int. Cl. A41D 13/05

(52) U.S. Cl. CPC A41D 13/05; A41D 13/1161

(57) ABSTRACT

An ear protector hook system for attaching to a band attached to a mask body and a cannula having two flexible tubes with nasal prongs therebetween. The system includes two ear protector hooks each comprised of a hook shaped structure constructed of four sections attached to each other end to end. A slot extending the length of the hook shaped structure extends from an outer surface of the hook shaped structure to a circular inner bore extending the length of the hook shaped structure to receive either the band attached to a mask body or the flexible tubes attached to the nasal prongs.

### TECHNICAL FIELD OF THE INVENTION

The present invention relates to a face mask or cannula ear protector hook, and more particularly to a face mask ear protector hook or a cannula tube ear protector hook which can easily be secured thereto.

### BACKGROUND OF THE INVENTION

Face masks are typically secured to a person's face with a single, thin elasticized strap that fits around a person's head. The ends of the strap are typically secured to each side of the face mask. Straps of this type however, often secure the mask at an angle that results in ear abrasion and improper mask placement. Additionally, the strap easily slips out of position and frequent readjustment of the mask and strap are needed to establish proper, comfortable mask placement.

Ear abrasion is a common problem in persons who wear oxygen masks, especially if they wear them on a long-term basis. It results when the strap, often an elastic strap, rubs repeatedly against the same part of a person's ear. The part of the ear most commonly affected by ear abrasions is the top part, where the Strap is generally caught and comes to rest, after it has slipped downwards out of its proper position. The abrasion on the top of the ear can be so severe as to actually result in the formation of a crevice or crack in which the strap rests and continues to cause damage.

Nursing staff often make futile efforts to comfort patients and prevent further damage to the ears, for example by readjusting the strap or by the application of Duoderm™ (an adhesive hydroactive dressing) to the affected area, which functions to promote epithelialisation of existing skin injuries and reduces contact of this area with the strap, acting prophylactically for the prevention of further friction damage. Patients who are unable to adjust the strap themselves are particularly prone to ear abrasion, as busy hospital Staff, family or friends are not always able to 0readjust the strap if it is causing damage to the ears. Given the frequency of ear abrasions in persons who wear oxygen masks, there is a need for an apparatus for use in preventing ear abrasion in persons who wear these masks.

Nasal cannulas are used to deliver oxygen to people who don't otherwise get enough of it. They are commonly used to provide relief to people with respiratory disorders Nasal cannulas and face masks are typically used to treat people who have respiratory conditions such as: asthma, bronchopulmonary dysplasia, or underdeveloped lungs in newborns, chronic obstructive pulmonary disease (COPD), cystic fibrosis, pneumonia, sleep apnea, and people in hospitals who have experienced trauma or acute conditions such as heart failure.

A nasal cannula consists of a narrow flexible plastic tubing used to deliver oxygen through the nostrils of patients using nasal breathing. The nasal tube includes two prongs that go inside the nostrils. Nasal cannulas are often secured over one or both the ears of the patient and as with the masks, discussed hereinbefore, can cause ear abrasion, especially for persons who wear nasal cannulas on a long-term basis. It results when the plastic tube rubs repeatedly against the same part of a person's ear. The part of the ear most commonly affected by ear abrasions behind the outer, curved flap part leading to the earlobe. This is the location where the plastic tubing of the cannula is generally caught and comes to rest. The abrasion or chaffing on the rear surface of the curved flap of the ear can be so severe as to actually result in the formation of a crevice or crack in which the cannula rests and continues to cause damage.

### SUMMARY OF THE INVENTION

According to an embodiment of the present invention, there is disclosed an ear protector hook system for attaching to a band attached to a mask body. The system includes two ear protector hooks. Each of the ear protector hooks is comprised of a hook shaped structure constructed of four sections attached to each other end to end. A first section of the four sections is shaped to hook over the top of a wearer's ear and rest against the head of the wearer. A third section of the four sections is adapted to rest in the crevice between the wearer's ear and the head of the wearer and exerting a force causing a second section between the first section and the third section to be pulled forward toward the mask. A fourth section of the four sections is connected to the third section and adapted to rest in the crevice between the wearer's ear and the head of the wearer so as to stabilize the mask body and prevent repeated rubbing of the ear protector against the same part of a person's ear. A slot extending the length of the single hook shaped structure which extends from an outer surface of the hook shaped structure to a circular inner bore extending the length of the single hook shaped structure receives the band attached to a mask body.

According to another embodiment of the present invention, there is disclosed an ear protector hook system for attaching a cannula to a wearer. The cannula has two flexible tubes with nasal prongs therebetween adapted to be placed around the ears of a wearer. The system includes two ear protector hooks. Each of the ear protector hooks is comprised of a hook shaped structure constructed of four sections attached to each other end to end. A first section of the four sections is shaped to hook over the top of a wearer's ear and rest against the head of the wearer. A third section of the four sections is adapted to rest in the crevice between the wearer's ear and the head of the wearer and exerts a force causing a second section between the first section and the third section to be pulled forward toward the nasal prongs. A fourth section of the four sections connected to the third section and adapted to rest in the crevice between the wearer's ear and the head of the wearer so as to stabilize the nasal prongs and prevent repeated rubbing of the ear protector against the same part of a person's ear. A slot extending the length of the single hook shaped structure extends from an outer surface of the hook shaped structure to a circular inner bore extending the length of the single hook shaped structure receives one of the two flexible tubes of the cannula.

### BRIEF DESCRIPTION OF THE DRAWINGS

The structure, operation, and advantages of the present invention will become further apparent upon consideration of the following description taken in conjunction with the accompanying figures (Figs.). The figures are intended to be illustrative, not limiting. Certain elements in some of the figures may be omitted, or illustrated not-to-scale, for illustrative clarity. The cross-sectional views may be in the form of “slices”, or “near-sighted” cross-sectional views, omitting certain background lines which would otherwise be visible in a “true” cross-sectional view, for illustrative clarity.

In the drawings accompanying the description that follows, both reference numerals and legends (labels, text descriptions) may be used to identify elements. If legends are provided, they are intended merely as an aid to the reader, and should not in any way be interpreted as limiting.

FIG. 1 is a side, three dimensional view of an ear protector hook, in accordance with the present invention.

FIG. 2 is a top, three dimensional exploded view of an ear protector hook secured to a string type elastic of a mask worn by an individual, in accordance with the present invention.

FIG. 3 is a side view of an ear protector hook secured to both sides of a string type elastic of a mask worn by an individual, in accordance with the present invention.

FIG. 4 is a side, three dimensional view of an ear protector hook secured to both sides of a string type elastic of a mask worn by an individual, in accordance with the present invention.

FIG. 5 is a rear three dimensional view of an ear protector hook secured to a string type elastic of a mask worn by an individual, in accordance with the present invention.

FIG. 6 is a side, three dimensional view of an ear protector hook secured to a canula worn by an individual, in accordance with the present invention.

FIG. 7 is a side, three dimensional view of an ear protector hook secured to a canula to be worn by an individual, in accordance with the present invention.

FIG. 8 is a side, three dimensional view of two protector hooks secured to a canula to be worn by an individual, in accordance with the present invention.

### DETAILED DESCRIPTION OF THE PREFERRED EMBODIMENTS

In the description that follows, numerous details are set forth in order to provide a thorough understanding of the present invention. It will be appreciated by those skilled in the art that variations of these specific details are possible while still achieving the results of the present invention. Well-known processing steps are generally not described in detail in order to avoid unnecessarily obfuscating the description of the present invention.

In the description that follows, exemplary dimensions may be presented for an illustrative embodiment of the invention. The dimensions should not be interpreted as limiting. They are included to provide a sense of proportion. Generally speaking, it is the relationship between various elements, where they are located, their contrasting compositions, and sometimes their relative sizes that is of significance.

In the drawings accompanying the description that follows, often both reference numerals and legends (labels, text descriptions) will be used to identify elements. If legends are provided, they are intended merely as an aid to the reader, and should not in any way be interpreted as limiting.

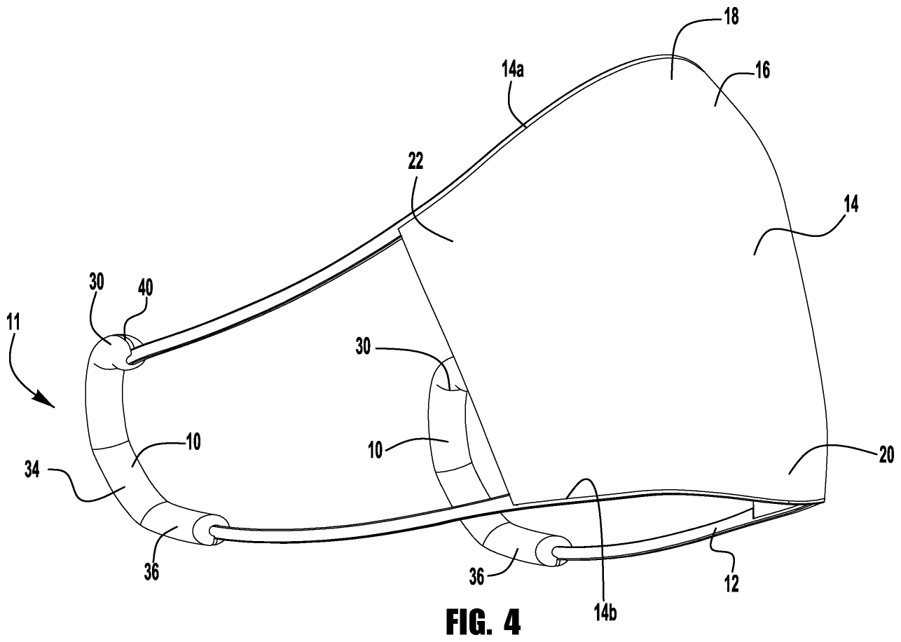

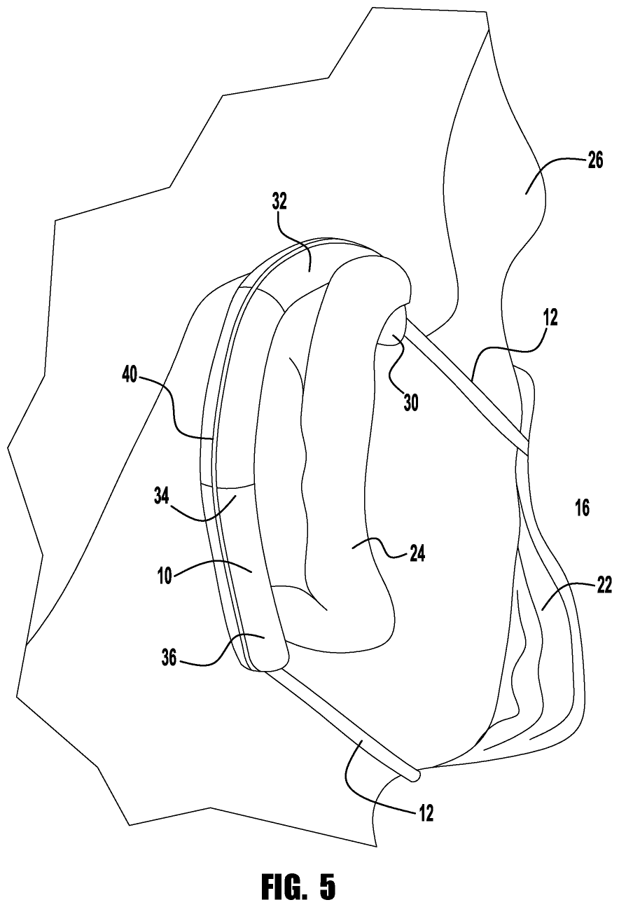

As shown in FIG. 1, there is illustrated a side, three dimensional view of the ear protector hook system 11, consisting of two identical ear protector hooks 10 for attaching, as shown in FIG. 4, to a band 12 attached along the upper edge 14a and lower edge 14b of a mask 14 by means of sewing, gluing or other fastener 16 and loops around the ear of the wearer. Typically, the band is formed from a single, thin elasticized strap. Before looping around a user's ears, ear protector hooks 10 are secured onto the band 12 which extends to the front of the mask 14. It can be appreciated that the band 12 should be sized for the wearer or, in accordance with the present invention.

FIG. 4 shows a mask 14 which includes a cup-like mask body 16 typically made of fibrous filter material and molded to fit over the mouth and nose of a wearer, generally following the contour of the wearer's face. The mask body 16 includes an upper portion 18 and a lower portion 20 as well as side portions 22, as shown more clearly in FIG. 4. A nose clip, not shown, can be utilized to provide additional forming over the bridge of the wearer's nose. Fabric-type fibrous filtering material of the mask body 16 removes particulates from the air, providing a breathable air supply. As shown in FIG. 1, a band 12 attaches at an upper point 14a by means of staple or other type of fastener and loops around the ear 24 of the wearer 26. After looping around the ear 24, the band 12 extends to the front of the mask 14 and extends around the opposite ear of the wearer. It can be appreciated that the band 12 should be sized for the wearer or may be adjustable or should include at least some elastic material to provide a snug fit.

It is understood that a snug fit is necessary to keep the mask in place over the mouth and nose of a wearer. However, the elastic band 12, typically being constructed of a single, thin elasticized strap, while being effective to keep the mask in place against the face of the wearer tends to cause ear abrasion and improper mask placement. Additionally, the strap 12 easily slips out of position and frequent readjustment of the mask and strap are needed to establish proper, comfortable mask placement.

Ear abrasion is a common problem in persons who wear masks, especially if they wear them on a long-term basis. It results when the strap 12, often an elastic strap, rubs repeatedly against the same part of a person's ear. The part of the ear most commonly affected by ear abrasions is the top part, where the strap is generally caught and comes to rest, after it has slipped downwards out of its proper position. The abrasion on the top of the ear can be so severe as to actually result in the formation of a crevice or crack in which the strap rests and continues to cause damage.

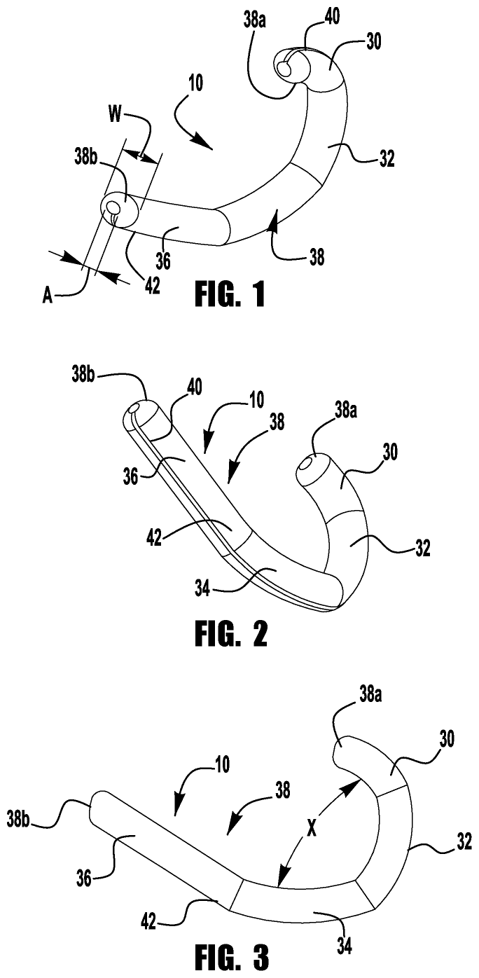

The ear protector hook 10, as shown in FIGS. 1-3, can be constructed of four sections 30,32, 34, and 36 (30-36) which are integrated into a single hook shaped structure 38. The first end 30 of the hook shaped structure 38 is designed to hook over the top of the ear and rest against the head 26 of the wearer. As discussed in more detail below, a slot 40 extends the length of the back or dorsal side 42 of the single hook shaped structure 38. The slot 40 has a width of about 0.6 mm to 1.0 mm and preferably 0.8 mm which extends from the outer surface of the structure 38 to a circular inner bore A having an inner diameter of about 4 mm to 5 mm and preferably about 3 mm. The structure 38 has a width W of about 6 mm to 10 mm and preferably about 8 mm to receive the strap 12. As shown in FIGS. 4 and 5, the strap exiting the slot 40 causes the first section 30 to press downwards against the top of the ear 24 and the second section 32 to press against the crevice formed between the top of the ear and the user's head. The taper of the upper end 30 and the second section 32 tends to optimize the grip and strength to hold the mask 14 in place.

The third section 34 of the ear protector hook 10 rests in the crevice between the ear 24 and the head of the wearer and exerts a force causing the second section 32 to be pulled forward toward the mask body 16. Being that the third section 34 pulled directly towards the front of the person's face, this portion tends to prevent repeated rubbing of the ear protector against the same part of a person's ear.

The fourth section 33 of the ear protector hook 10 also rests in the crevice between the ear 24 and the head of the wearer and tends to stabilize the mask body 16 and prevent repeated rubbing of the ear protector against the same part of a person's ear.

As shown in FIG. 3, the angle x between the first section 30 and the section 34 is between 80 and 100 degrees and preferably about 85 to 95 degrees and most preferably about 90 degrees. The angel x has been determined to grip about most ear sizes making the ear protector hook easy to place on the ear while securely holding the mask in place. The ear protector hook 10 is constructed of a relatively, non-flexible material, such as for example a fully cured ethylene propylene diene monomer (EPDM) rubber in a thermoplastic matrix of polypropyle (PP) like Santropene™, a material selected from the group consisting of a fully cured ethylene propylene diene monomer (EPDM) rubber in a thermoplastic matrix of polypropyle (PP), a high performance thermoplastic elastomer and silicone so that it doesn't move around on the ear once it is in place.

In use, the ear protector hook 10 is secured to the strap 12 by inserting the strap into the slot 40 which extends around the length of the back or dorsal side 42 of the single hook shaped structure 38. The slot 40 is open at either end 38a and 38b, respectively, of the hook shaped structure 38 so that the strap 12 extends out of the open ends so that the mask 14 can be comfortably fit to the user's face.

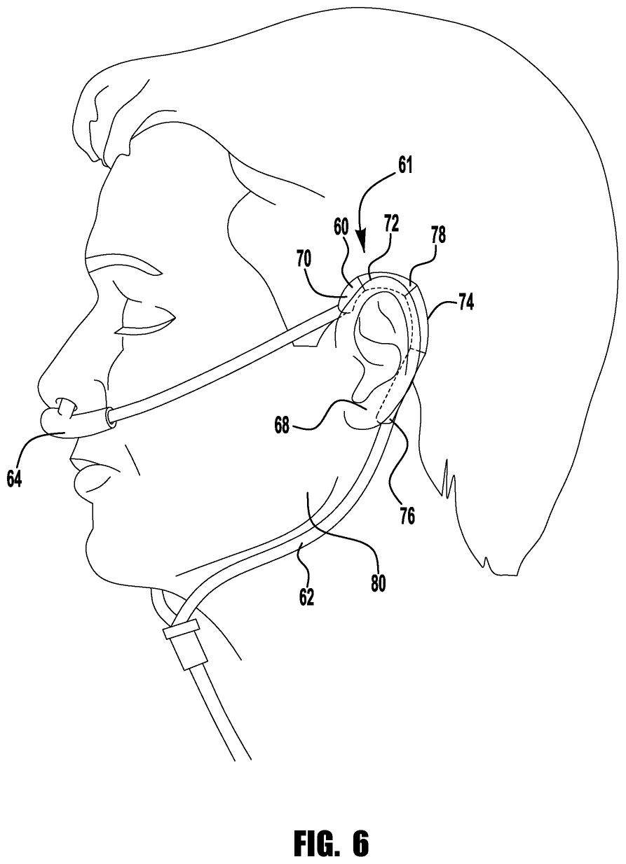

As shown in FIG. 6, there is illustrated a side, three dimensional view of an ear protector hook system 61, consisting of two ear protector hooks 60 for attaching a cannula 63 around the ear of the wearer. Typically, the cannula 63 is formed from a hollow surgical tube that is flexible and can be inserted into a person's body for the delivery or removal of a fluid or gathering samples. The cannula 63 shown in FIG. 8 includes a flexible tube or hose 62 with nasal prongs 64. The flexible tube 62 can be include two separate tubes 62a and 62b which have one end inserted into the nasal prongs 64 and the opposite ends into a connector 66. Before looping around a user's ears, ear protector hooks 60 are secured onto the flexible tube or hose 62. The ear protector hook 60 is constructed of a relatively, non-flexible material, a material selected from the group consisting of a fully cured ethylene propylene diene monomer (EPDM) rubber in a thermoplastic matrix of polypropyle (PP), a high performance thermoplastic elastomer and silicone so that it doesn't move around on the ear once it is in place, as shown in FIG. 6.

It is understood that a snug fit is necessary to keep the nasal prongs 64 in place within the nose of a wearer. However, the cannula being constructed of a single, flexible hose, while being effective to keep the nasal prongs 64 in place within the nostrils of the wearer tends, the flexible cannula tube can move about on the wearer's ears and cause ear abrasion.and improper placement of the nasal prongs. Additionally, while the cannula 62 is flexible and can cause the nasal prongs 64 to slip out of position. The ear protector hook 60, being relatively inflexible, maintains its position on the wearer's ears and frequent readjustment of the cannula and nasal prongs are not needed to establish proper and comfortable placement.

Ear abrasion is a common problem in persons who wear cannula, especially if they wear them on a long-term basis. It results when the flexible cannula tube or hose 62 rubs repeatedly against the same part of a person's ear 68. The part of the ear most commonly affected by ear abrasions is the top part, where the cannula hose is generally caught and comes to rest, after it has slipped downwards out of its proper position. The abrasion on the top of the ear can be so severe as to actually result in the formation of a crevice or crack in which the cannula tube rests and continues to cause damage.

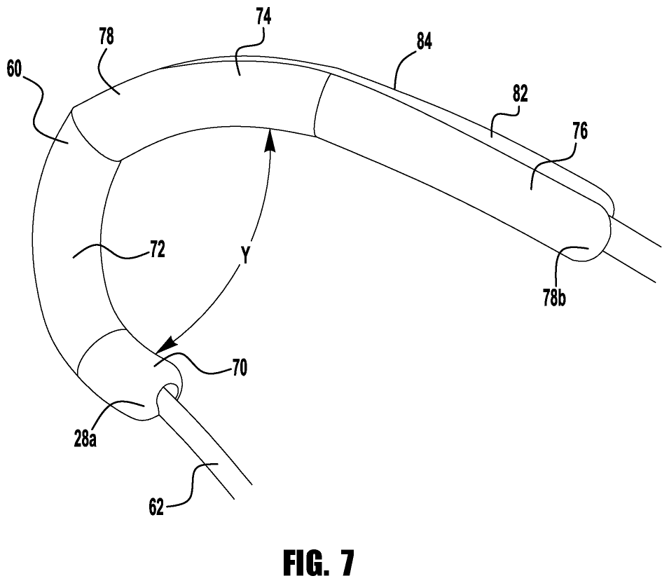

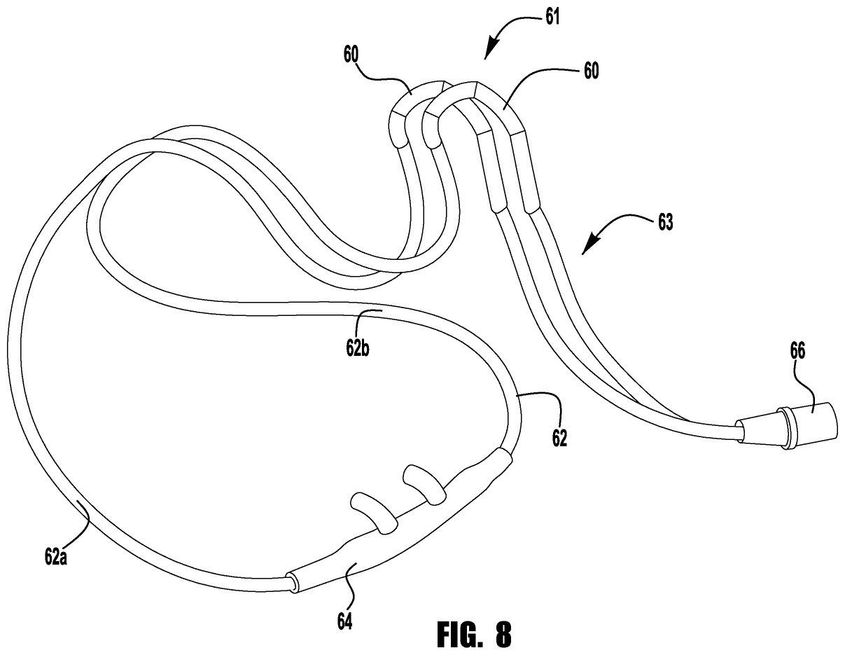

The ear protector hook 60, as shown in FIG. 7, can be constructed of four sections 70,72, 74, and 76 (70-76) which are integrated into a single hook shaped structure 78. The outer end 70 of the hook shaped structure 78 is designed to hook over the top of the ear and rest against the head 80 of the wearer. As discussed in more detail below, a slot 82 extends the length of the back or dorsal side 84 of the single hook shaped structure 78 and has a width of about 6 mm to 10 mm and preferably 8 mm to receive the cannula tube 62. The slot 82 extends from the outer surface of the structure 78 to a circular inner bore having an inner diameter of 2 mm to about 4 mm and preferably about 3 mm.

As shown in FIG. 6, the tube 62 exiting the slot 82 causes the first section 60 to press downwards against the top of the ear 68 and the second section 72 to press against the crevice formed between the top of the ear and the user's head. The taper of the upper end 70 and the second section 72 tends to optimize the grip and strength to hold the prongs 64 in place.

The third section 74 of the ear protector hook 60 rests in the crevice between the ear 68 and the head of the wearer and exerts a force causing the second section 72 to be pulled forward toward the nasal prongs 64. Being that the third section 74 is pulled directly towards the front of the person's face, this portion tends to prevent repeated rubbing of the ear protector against the same part of a person's ear.

The fourth section 76 of the ear protector hook 60 also rests in the crevice between the ear 68 and the head 80 of the wearer and tends to stabilize the nasal prongs 64 and prevent repeated rubbing of the ear protector hook 60 against the same part of a person's ear.

As shown in FIG. 7, the angle y between the first section 70 and the third section 74 is between 80 and 100 degrees and preferably about 85 to 95 degrees and most preferably about 90 degrees. The angel y has been determined to ensure that the ear protector hook 60 grips about most ear sizes making the ear protector hook easy to place on the ear while securely holding the nasal prongs 64. The structure 78 has a width of about 6 mm to 10 mm and preferably 8 mm to receive the cannula hoses 62.

In use, the ear protector hook 60 is secured to the two flexible cannula hoses 62a and 62b by inserting the hoses into the slot 82 which extends around the length of the back or dorsal side 84 of the single ear protector hook shaped structure 78. The slot 82 is open at either end 78a and 78b, respectively, of the hook shaped structure 78 so that the cannula hose 62 extends out of the open ends so that the nasal prongs 64 can be comfortably fit to the user's face.

Although the invention has been shown and described with respect to a certain preferred embodiment or embodiments, certain equivalent alterations and modifications will occur to others skilled in the art upon the reading and understanding of this specification and the annexed drawings. In particular regard to the various functions performed by the above described components (assemblies, devices, etc.) the terms (including a reference to a “means”) used to describe such components are intended to correspond, unless otherwise indicated, to any component which performs the specified function of the described component (i.e., that is functionally equivalent), even though not structurally equivalent to the disclosed structure which performs the function in the herein illustrated exemplary embodiments of the invention. In addition, while a particular feature of the invention may have been disclosed with respect to only one of several embodiments, such feature may be combined with one or more features of the other embodiments as may be desired and advantageous for any given or particular application.

1. An ear protector hook system for attaching to a band attached to a mask body, comprising:
two ear protector hooks;
each of the ear protector hooks comprised of a hook shaped structure constructed of four sections attached to each other end to end;
a first section of the four sections shaped to hook over the top of a wearer's ear and rest against the head of the wearer;
a third section of the four sections adapted to rest in the crevice between the wearer's ear and the head of the wearer and exerting a force causing a second section between the first section and the third section to be pulled forward toward the mask;
a fourth section of the four sections connected to the third section and adapted to rest in the crevice between the wearer's ear and the head of the wearer so as to stabilize the mask body and prevent repeated rubbing of the ear protector hook against the same part of a person's ear;
a slot extending the length of the hook shaped structure which extends from an outer surface of the hook shaped structure to a circular inner bore extending the length of the hook shaped structure receiving the band attached to a mask body.

2. The ear protector hook system of claim 1 wherein the slot extends the length of the back side of the hook shaped structure.

3. The ear protector hook system of claim 2 wherein the slot extends from an outer surface of the hook shaped structure to a circular inner bore extending the length of the back side of the hook shaped structure.

4. The ear protector hook system of claim 2 wherein the slot has a width of about 0.6 mm to about 1.0 mm which extends from the outer surface of the hook shaped structure to a circular inner bore.

5. The ear protector hook system of claim 4 wherein the inner bore has an inner diameter of about 3 mm to receive the strap.

6. The ear protector hook system of claim 5 wherein the strap exiting the slot causes the first section to press downwards against the top of the wearer's ear and the second section to press against the crevice formed between the top of the wearer's ear and the user's head.

7. The ear protector hook system of claim 6 wherein the strap is attached along the upper edge and lower edge of to a mask and loops around the ears of the wearer.

8. The ear protector hook system of claim 7 wherein the band is formed from an elasticized strap.

9. The ear protector hook system of claim 8 wherein the ear protector hooks are secured onto a section of the strap which extends between the ear of the wearer to the front of the mask.

10. The ear protector hook system of claim 1 wherein the angle x between the first section and the third section is between 85 to 95 degrees.

11. The ear protector hook system of claim 1 wherein the angle x between the first section and the third section is 90 degrees.

12. The ear protector hook system of claim 1 wherein the ear protector hook is constructed of a relatively, non-flexible material constructed of a material selected from the group consisting of a fully cured ethylene propylene diene monomer (EPDM) rubber in a thermoplastic matrix of polypropyle (PP), a high performance thermoplastic elastomer and silicone so that it doesn't move around on the ear once it is in place.

13. An ear protector hook system for attaching a cannula having two flexible tubes with nasal prongs therebetween, around the ears of a wearer, comprising:
two ear protector hooks;
each of the ear protector hooks comprised of a hook shaped structure constructed of four sections attached to each other end to end;
a first section of the four sections shaped to hook over the top of a wearer's ear and rest against the head of the wearer;
a third section of the four sections adapted to rest in the crevice between the wearer's ear and the head of the wearer and exerting a force causing a second section between the first section and the third section to be pulled forward toward the nasal prongs;
a fourth section of the four sections connected to the third section and adapted to rest in the crevice between the wearer's ear and the head of the wearer so as to stabilize the mask body and prevent repeated rubbing of the ear protector against the same part of a person's ear;
a slot extending the length of the hook shaped structure which extends from an outer surface of the hook shaped structure to a circular inner bore extending the length of the hook shaped structure receiving one of the two flexible tubes of the cannula.

14. The ear protector hook system of claim 13 wherein the angle y between the first section and the third section is between 85 to 95 degrees.

15. The ear protector hook system of claim 13 wherein the ear protector hook is constructed of a relatively, non-flexible material selected from the group consisting of a fully cured ethylene propylene diene monomer (EPDM) rubber in a thermoplastic matrix of polypropyle (PP), a high performance thermoplastic elastomer and silicone so that it doesn't move around on the ear once it is in place

16. The ear protector hook system of claim 15 wherein the slot extends the length of the back side of the hook shaped structure.

17. The ear protector hook system of claim 16 wherein the slot extends from an outer surface of the hook shaped structure to a circular inner bore extending the length of the hook shaped structure.

18. The ear protector hook system of claim 17 wherein the slot has a width of about 0.6 mm to 1.0 which extends from the outer surface of the hook shaped structure to a circular inner bore to receive the flexible tubes.

19. The ear protector hook system of claim 18 wherein the inner bore has an inner diameter of about 2 mm to about 4 mm to receive the flexible tubes.

20. The ear protector hook system of claim 19 wherein the ear protector hooks are secured onto one of the two flexible tubes of the cannula which extends between the ears of the wearer to the nasal prongs.
